(19) United States
(12) Patent Application Publication
(10) Pub. No.: US 2009/0216205 A1
(43) Pub. Date: Aug. 27, 2009

(54) FLUID WASTE COLLECTION AND DISPOSAL SYSTEM AND METHOD

(75) Inventors: Marshall C. Ryan, Grantsburg, WI (US); Kevin R. Davidson, Lakeville, MN (US)

Correspondence Address: PATENT DEPARTMENT;LARKIN, HOFFMAN, DALY & LINDGREN, LTD., 1500 WELLS FARGO PLAZA, 7900 XERXES AVENUE SOUTH, BLOOMINGTON, MN, 55431, US

(73) Assignee: BioDrain Medical, Inc., Mendota Heights, MN (US)

(21) Appl. No.: 12/404,189

(22) Filed: Mar. 13, 2009

Related U.S. Application Data
(63) Continuation-in-part of application No. 12/277,985, filed on Nov. 25, 2008.
Application No. 12/277,985 is a division of application No. 10/524,086, filed on Feb. 9, 2005, now Pat. No. 7,469,727.

(51) Int. Cl. A61M 1/00

(52) U.S. Cl. 604/319

(57) ABSTRACT

A system and method for continuously aspirating and collecting fluid waste during a medical procedure without interruption for disposal of the collected fluid waste. The system includes a first reservoir in communication with a vacuum source which produces a negative pressure therein to draw the fluid waste from the patient using a suction hose connected thereto. A second reservoir is in communication with the first reservoir. A pump draws fluid from the first and second reservoirs while additional fluid waste continues to be drawn into the first reservoir through the fluid inlet.

### CROSS-REFERENCE TO RELATED APPLICATIONS

This is a continuation-in-part of U.S. patent application Ser. No. 12/277,985 filed Nov. 25, 2008, which is a Divisional of U.S. patent application Ser. No. 10/524,086, now U.S. Pat. No. 7,469,727, which claims the benefit of PCT Application No. PCT/US2003/025018 (Pub. No. WO2004/018295) filed Feb. 5, 2005.

### BACKGROUND

Systems for collecting and disposing of bodily fluids and other fluids that are aspirated from a patient during surgical procedures are well known. Conventional fluid waste collection systems typically use some type of container or canister into which the aspirated body fluids are accumulated. As the fluid collection canisters become filled during the course of a surgical procedure, the filled canisters are replaced with empty canisters. During the surgical procedure and or after the surgical procedure is completed, the fluid filled canisters are typically carted from the operating room to a central collection location for disposal or, alternatively, the canisters may be emptied, cleaned, and re-used.

The canisters into which the fluid is collected are typically transparent so the surgical team can visually assess the color of the aspirated fluid as an indicator of the amount of blood loss occurring. Furthermore, the canisters typically include graduated markings to allow the surgical team to estimate the volume of fluid loss from the patient during the procedure by comparing the volume of the collected fluid in relation to the known quantities of saline or other fluids introduced into the patient during the procedure so as to ensure that no excess fluid remains within the body cavity and to ensure that excessive blood loss has not occurred; both being conditions that may place the patient at an increased post-operative risk.

It should be appreciated that the aspirated fluids may be contaminated with pathogens, such as HIV, HPV, Hepatitis, MRSA and other infectious agents. Accordingly, handling of fluid collection canisters by hospital personnel when replacing the canisters during a surgical procedure or after the surgical procedure creates a risk that the handlers may come into contact with the contaminated bodily fluids contained in the canisters, for example, if the canister is dropped or spilled or if the canister leaks, and particularly if the handler is required to empty the filled canisters, because the fluid may splash onto the handler as it is being poured from the canister.

As disclosed in U.S. Pat. No. 5,242,434 issued to Terry, the containers may be partially pre-filled with a disinfectant to destroy any pathogens in the aspirated fluid as it enters the canisters, thereby minimizing risk to the handlers even if the canister is spilled. The disinfected fluid may then be pored down the sanitary sewer or otherwise disposed of. It is also known to add a solidify agent or coagulant into the canisters to minimize the potential for spillage, splashing and leakage. Using a solidifying agent or coagulant often increases the disposal costs as canisters with such an agent or coagulant are treated as hazardous waste and must be incinerated or delivered to a landfill. Under any of these methods, there remains a risk that handlers of the canisters will come into contact with the fluid waste. There is also the additional labor and cost associated with having to purchase, store, handle, disinfect and/or dispose of the canisters themselves.

Apart from the risk of exposure to the fluid waste when collecting the aspirated fluid in canisters, another disadvantage of systems that utilize canisters is that the surgical procedure may have to be interrupted to replace a filled canister with an empty canister. Accordingly, there remains a need for a fluid waste collection system that permits the collection of aspirated fluids without interruption to dispose of the collected aspirated fluid, which eliminates the need for handling of canisters and the potential risk of exposure to pathogens associated therewith.

### DESCRIPTION OF THE DRAWINGS

FIG. 1 is a perspective view of a preferred embodiment of a fluid waste collection and disposal system.

FIG. 2 is a side elevation view of the fluid waste collection and disposal system of FIG. 1.

FIG. 3 is a perspective view of the fluid waste collection and disposal system of FIG. 1 with the housing partially removed and partially in cross-section to illustrate the arrangement of the preferred components of the system.

FIG. 4 is a side elevation view of the fluid waste collection and disposal system of FIG. 1 with the housing partially removed and partially in cross-section to illustrate the arrangement of the preferred components of the system.

FIG. 5 illustrates a preferred touch screen window of the fluid waste collection and disposal system of FIG. 1.

FIG. 6 is a diagram illustrating fluid waste entering the first and second reservoirs with the fluid level shown at the low level sensor.

FIG. 7 is a schematic diagram illustrating fluid waste entering the first and second reservoirs with the fluid level shown at the high level sensor.

FIG. 8 is a schematic diagram illustrating fluid waste entering the first and second reservoirs while the fluid waste is also being pumped out of the reservoirs.

FIG. 9 is a schematic diagram illustrating the cleaning solution being drawn from the cleaning solution bottle into the first and second reservoirs; the cleaning solution level being shown at the maximum level sensor.

FIG. 10 is a schematic diagram illustrating the cleaning solution being recirculated by the pump and being sprayed into the first reservoir.

FIG. 11 is a schematic diagram illustrating the cleaning solution being pumped out of the reservoirs.

### DETAILED DESCRIPTION

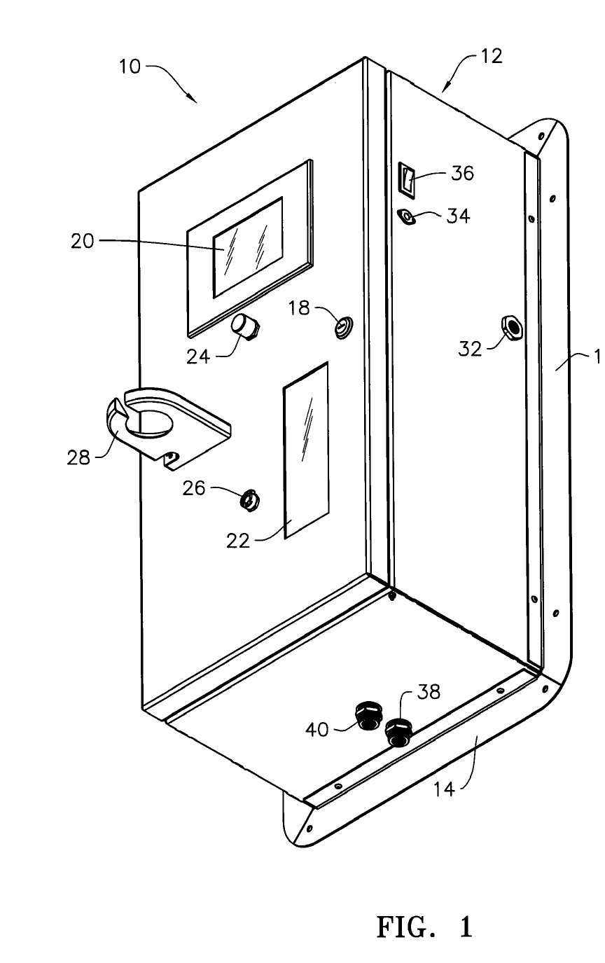

Referring now to the drawings, wherein like reference numerals designate identical or corresponding parts throughout the several views, FIG. 1 is a perspective view of a preferred embodiment of a fluid waste collection and disposal system designated generally by reference numeral 10. The system 10 is shown having a housing 12 preferably adapted for mounting on a wall or in a partially recessed fashion into a wall in the operating room or other facility in which fluid aspiration procedures are performed. A mounting flange 14 is preferably provided for securing the housing to any suitable surface or structure using appropriate fasteners. It should be appreciated, however, that the system 10 may be a free standing stationary or portable system, provided that, for reasons that will become evident later, a suitable vacuum source and power source is available as well as a sanitary sewer drain or the like into which the fluid waste may be discharged.

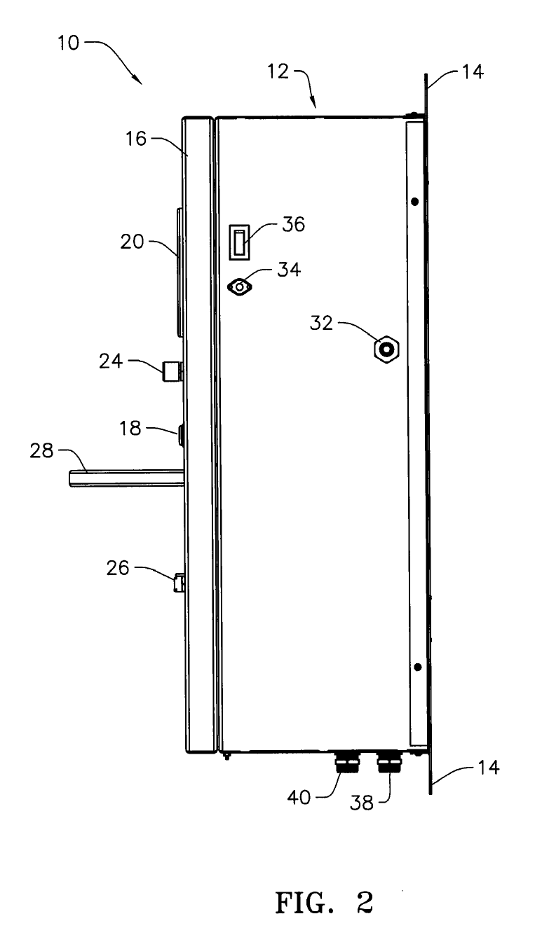

The housing 12 preferably includes a hinged front panel 16 for access to the interior of the housing and the components therein (discussed later). The front panel 16 preferably includes a lock 18 to prevent unauthorized access to the interior of the housing 12. The hinged front panel 16 preferably includes a touch screen display 20, a fluid viewing window 22, a vacuum adjustment 24, a suction port 26 and a cleaning solution hanger 28 for removably receiving a cleaning solution package 30. As best viewed in FIG. 2, one side panel of the housing 12 preferably includes a vacuum connection port 32, a power source connection 34 and an on-off switch 36. In a preferred embodiment of the system 10, the power source is preferably 24 Volt DC but any suitable power source may be utilized. A bottom panel of the housing 12 preferably includes a main drain port 38 and a secondary drain port 40. It should be appreciated that the particular location of the foregoing items may vary depending on where and how the system 10 is installed and/or mounted.

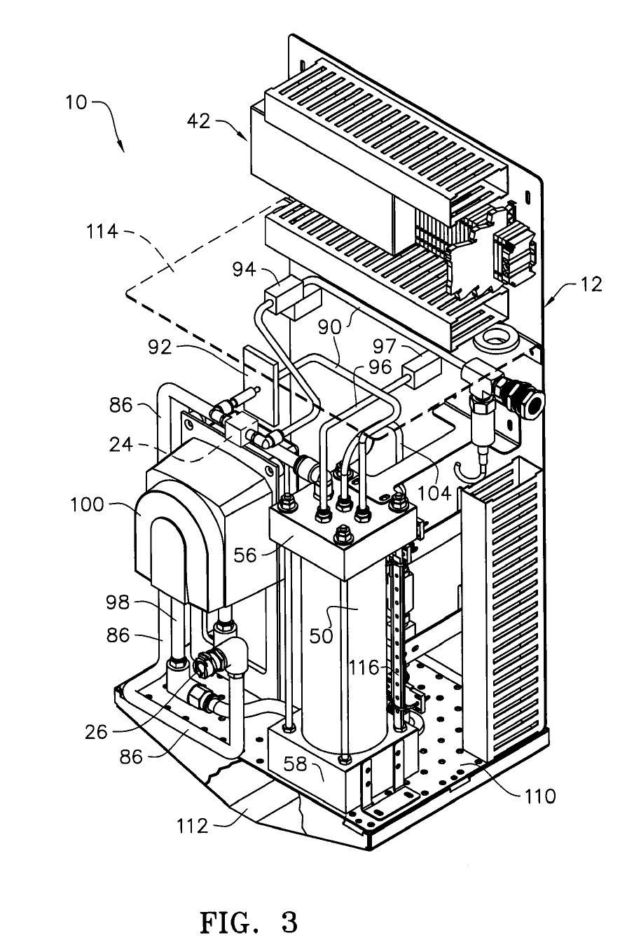

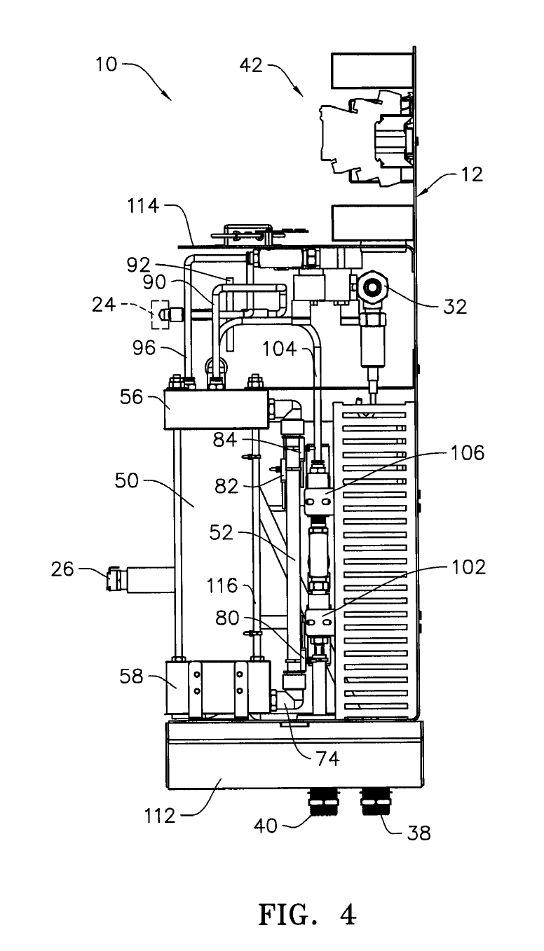

The system 10 preferably includes a programmable logic controller (“PLC”) (not shown) which interfaces with the touch screen display 20 and other circuitry, indicated generally by reference numeral 42 in FIGS. 3 and 4. Such circuitry and associated programming for the PLC for providing the features and performing the functions described below is readily understood and recognized by those skilled in the art and therefore further discussion on the circuitry is not warranted. Rather than using a PLC and associated circuitry, it should be appreciated that solid state circuitry could be utilized which could further reduce the total size of system 10 if desired as well as provide additional desired functionality.

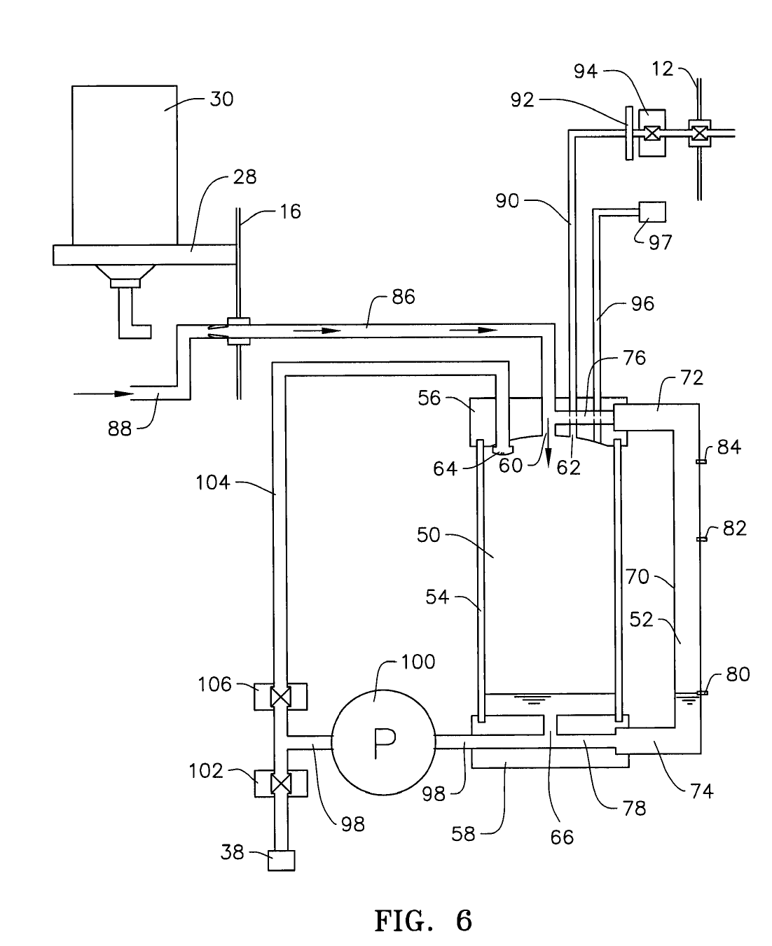

FIG. 3 is a perspective view and FIG. 4 is a right side elevation view of a preferred embodiment of the system 10 with the top and side panels of the housing 12 removed for better viewing of the interior components of the preferred embodiment of the system 10 as hereinafter described. FIGS. 6-10 are schematic diagrams illustrating the relationship and operation of those preferred components.

Accordingly, referring to FIGS. 3, 4 and 6, disposed within the interior of the housing 12, is a first reservoir 50 and a second reservoir 52. The first reservoir 50 is preferably a vertically oriented transparent cylinder 54, preferably made of transparent acrylic or other suitable material of sufficient thickness to safely withstand the negative pressures typically used for the central vacuum systems of a medical facility, which typically are not greater than 25 inches (635 mm) of mercury (Hg). Transparent material is preferred for the first reservoir 50 to allow the surgeon or other medical personnel to view the aspirated fluid as it is collected for assessing its color or other characteristics. Top and bottom end caps 56, 58 preferably seal the cylinder 54. Gaskets (not shown) preferably provide a fluid tight seal between the wall of the cylinder 54 and the end caps 56, 58. As best illustrated in FIG. 6, the top end cap 56 preferably includes a fluid inlet 60, a vacuum inlet 62, and a recirculation inlet 64. The bottom end cap 58 preferably includes a fluid outlet 66.

The second reservoir 52 is also preferably a vertically oriented transparent cylinder 70. As best illustrated in FIG. 6, the second reservoir 52 is fluidly connected to the first reservoir 50 preferably via horizontal top and bottom conduits 72, 74 connected to transverse bores 76, 78 in the top and bottom end caps 56, 58. Preferably disposed along the length of the vertically oriented cylinder 70 is a low fluid level sensor 80, a high fluid level sensor 82 and a maximum fluid level sensor 84. The purpose of the fluid level sensors will be discussed later. The preferred sensors are capacitive proximity sensors, but other sensors may be equally suitable.

A fluid line 86 connects the fluid inlet 60 of the first reservoir 50 to the suction port 26 on the front panel 16 of the housing 12. On the exterior of the housing 12, a suction hose 88 connects to the suction port 26. An end effector (not shown) on the distal end of the suction hose 88, is used to suction or aspirate the waste fluid from the patient. If desired, a multi-port manifold may be connected to the suction port 26 to enable multiple suction hoses 88 to be connected to the system 10 at one time. The suction hose(s) 88 and the multi-port manifold are preferably disposable and are preferably discarded after a single use.

A vacuum line 90 extends between the vacuum port 32 and the vacuum inlet 62 in the top end cap 56 of the first reservoir 50. A vacuum protection filter 92, such as a Pall filter (Part Number 8001052) or a ZenPure filter (Part Number G50RF0201N1), is preferably coupled to a solenoid-operated vacuum interrupt valve 94. If waste fluid for some reason backs up into the vacuum line 90 such that it comes into contact with the filter 92, the vacuum protection filter 92 is preferably designed to swell so as to prevent the passage of fluid and air through the filter, thereby causing the vacuum interrupt valve 94 to actuate interrupting the vacuum until the filter 92 is replaced. A pressure relief line 96 is also in communication with the first reservoir 50 through the top end cap 56. A solenoid-operated pressure relief valve 97 is disposed to actuate to vent the reservoirs 50, 52 to atmosphere in the event excess negative pressure builds up in the first and second reservoirs 50, 52.

A drain line 98 is in fluid communication with the fluid outlet 66 in the bottom end cap 56 and transverse bore 78 of the first reservoir 50. A pump 100 is disposed along the drain line 98 between the fluid outlet and the main drain port 38. The main drain port is preferably connected to a pipe (not shown) for discharging the fluid waste into the facility's sanitary sewer system. Also preferably disposed along the main drain line 98 between the pump 100 and the main drain port 38, is a normally closed solenoid-operated drain valve 102. The pump 100 is preferably a peristaltic pump, but may be any suitable positive displacement pump and/or metering pump capable of measuring the volume of fluid pumped. It is to be understood that as used herein, all peristaltic pumps are considered to be a type of positive displacement pump, and all positive displacement pumps are considered to be a type of metering pump. However, in addition to peristaltic pumps there are other types of positive displacement pumps known to those of skill in the art which may be equally suitable for the system 10. Furthermore, in addition to positive displacement pumps, there are other types of metering pumps known to those of skill in the art which may be equally suitable for the system 10.

A recirculation line 104 preferably tees from the drain line 98 upstream of the drain valve 102, but downstream of the pump 100. A normally closed solenoid-operated recirculation valve 106 is preferably disposed along the recirculation line 104 where it tees off from the drain line 98. The recirculation line 104 connects to the recirculation inlet 64 in the top end cap 54 of the first reservoir 50.

Referring back to FIGS. 3-4, the interior base of the housing 12 includes a perforated plate 110 disposed over a tray 112 such that any fluid waste from any fluid leaks in the system components will drain into the bottom of the tray 112. The bottom of the tray 112 preferably slopes from side-to-side and from front to back to direct any such spilled or leaking fluid toward the secondary drain port 40. The secondary drain port 40 is also preferably connected to the facility's sanitary sewer lines. The system 10 preferably includes a sensor (not shown) disposed near the secondary drain port such that when fluid waste comes into contact with the sensor a signal is generated to set off an alarm condition to notify the operator that a fluid leak has occurred. A buffer plate 114 preferably separates the system's electrical components from the fluid filled components to prevent or minimize potential damage to the electrical components in the event of a leak.

Continuing to refer to FIGS. 3-4, a light strip 116, preferably comprising a plurality of white light emitting diodes (LEDs), is disposed behind the first reservoir 50 to back-light the fluid in the reservoir 50 so it can be better viewed through the viewing window 22 in the front panel 16. If there is an alarm condition, for example if there is a leak or if the vacuum has been interrupted due to fluid back-up, the LEDs are preferably caused to light and flash to visually indicate an alarm condition. Under any alarm condition, the PLC is preferably programmed to flash an error message on the touch screen display 20.

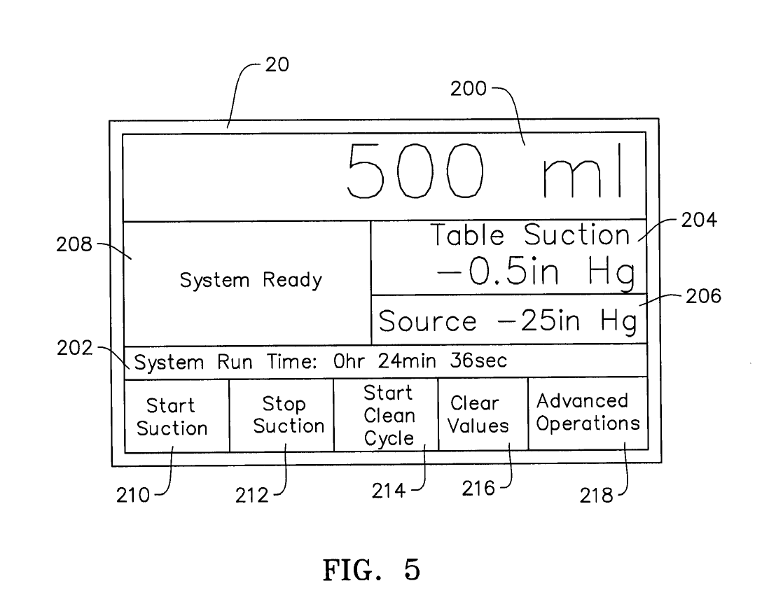

In FIG. 5, a preferred display screen is illustrated for the touch screen display 20. As illustrated, the preferred touch screen display 20 includes a “Fluid Pumped” display 200, a “System Run Time” display 202, a “Table Suction” display 204, a “Source Suction” display 206, a status/information window 208, and a plurality of selectable operational functions, including a “Start Suction” selection 210, a “Stop Suction” selection 212, a “Start Clean Cycle” selection 214, a “Clear Values” selection 216 and an “Advanced Operations” selection 218. The Fluid Pumped display 200 identifies the volume of fluid pumped (preferably in milliliters) since pressing the Start Suction selection 210. The System Run Time display 202 identifies the time passed, preferably displayed in hours, minutes and seconds, since pressing the Start Suction selection 210. The Table Suction display 204, preferably in inches or mm Hg, identifies the negative pressure at the suction port 26 which is controlled by the vacuum adjust dial 24 on the front panel 16. The Source Suction display 206, preferably in inches or mm Hg, identifies the suction provided by the facility's vacuum system to which the vacuum port 32 is connected. The status/information window 208 provides information to the operator such as the current operation selection, system status or any alarm conditions.

In operation, the system 10 is turned on by pressing the on-off rocker switch 36 to the “On” position. The touch screen 20 is activated and preferably displays a “system ready” message in the status/information window 208 to indicate to the operator that the system is ready for operation. Upon selecting the Start Suction operation 210, the vacuum interrupt valve 94 is opened permitting communication of the vacuum source with the first reservoir 50 through the vacuum lines 90. The operator may adjust the amount of Table Suction 204 using the vacuum adjust dial 24. The operator then attaches the suction tube(s) 88 to the suction port 26 and/or the manifold if used—it being understood, that the end effector (not shown) on the distal end of the suction tube 88 will also typically have a regulator for controlling the amount of negative pressure through the end effector. When the end effector on the suction hose 88 is place in contact with fluid, the fluid is caused to be drawn through the suction hose 88 and into the first reservoir 50. The pump 100 is not yet actuated and the solenoid-operated drain valve 102 remains closed. The fluid entering the first reservoir 50 is preferably visible through the window 22 in the front panel. As noted earlier, a light strip 116 is disposed to back-light the aspirated fluid accumulating in the first reservoir so it can be better viewed by the operator.

As illustrated in FIG. 6, because the first and second reservoirs are open to each other through the transverse bores 76, 78, the fluid inlet 60 and the fluid outlet 66, and thus are under the same negative pressure, the aspirated fluid accumulating in the first reservoir 50 flows through the fluid outlet 66 and into the second reservoir 52. It should be appreciated that as the aspirated fluid enters the first reservoir, the incoming air and fluid mixture can cause the accumulating fluid to be quite turbulent. However, as the fluid flows through the fluid outlet 66 in the bottom end cap 58, there is little if any turbulence of the fluid as it enters the second reservoir 52. Additionally, any foam in the accumulating fluid caused by the turbulence in the first reservoir will float on top of the fluid and thus, very little if any foam will pass into the second reservoir. Thus, the fluid in the second reservoir provides a more accurate measure of the accumulated fluid waste.

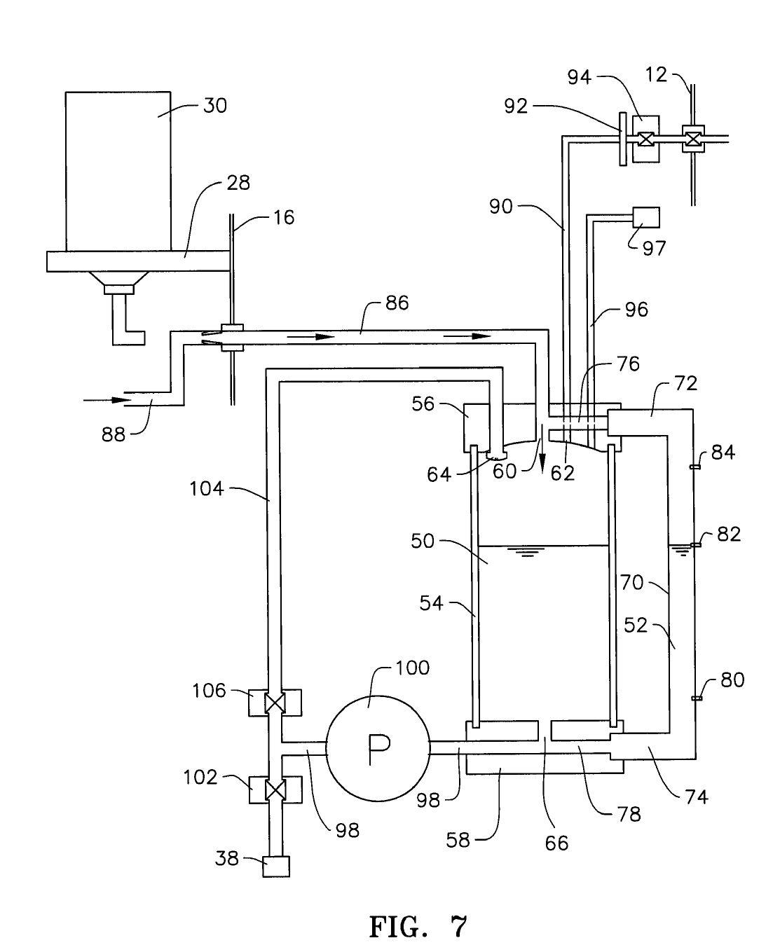

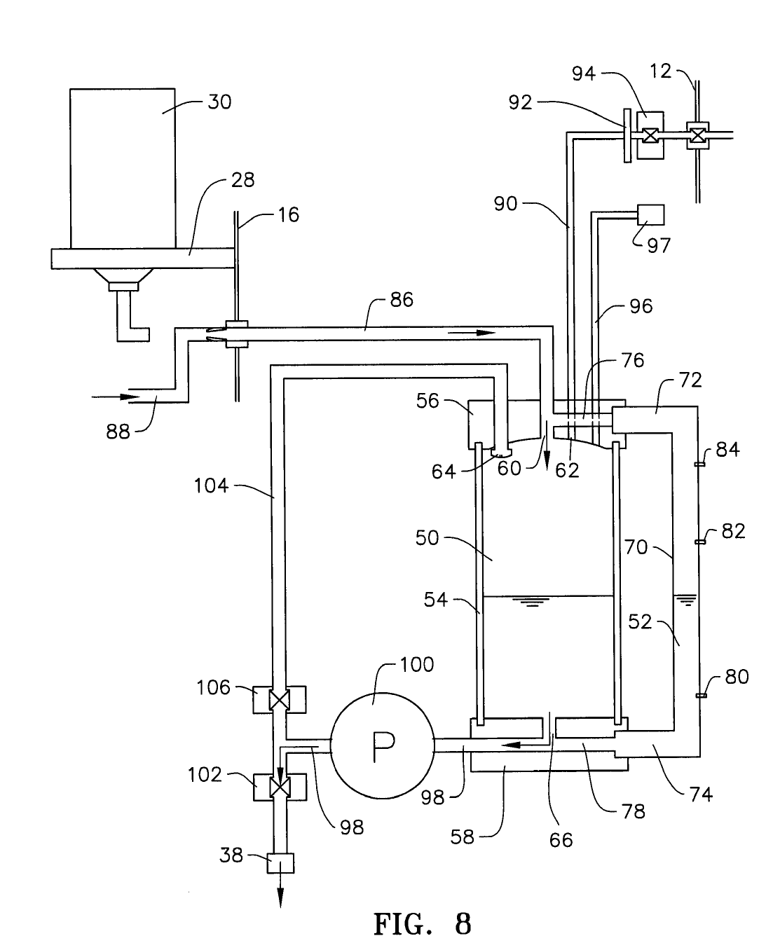

When the fluid level in the second reservoir 52 reaches the high level sensor 82 as illustrated in FIG. 7, the sensor 82 generates a signal causing the normally closed solenoid-operated drain valve 102 to open and to actuate the pump 100. As illustrated in FIG. 8, the actuation of the pump 100 draws the fluid waste from the first and second reservoirs 50, 52 through the fluid outlet 66 and transverse bore 78 and into the drain line 98 where it then passes out the drain port 38 and into the pipe connected to the facility's sanitary sewer. So that the fluid drains from both reservoirs 50, 52 at an equal rate, it may be necessary to provide a restriction in the fluid outlet 66, the transverse bore 78 and/or in the horizontal conduit 74. The need or amount of restriction may vary depending on the relative sizes and arrangement of the reservoirs and/or the size and positioning of the fluid outlet 66.

As illustrated in FIG. 8, the suctioning of the fluid is continuous and is uninterrupted during the entire medical procedure, even while the pump 100 pumps the accumulated fluid out of the reservoirs 50, 52. Because the discharge rate of the pump 100 is typically greater than the rate at which the fluid waste enters the first reservoir 50, the fluid level is quickly drawn down below the low level sensor 80, the absence of liquid on the low level sensor generates a signal stopping the pump 100 and causing the solenoid-operated drain valve 102 to close. When the fluid level again reaches the high level sensor 82, the presence of liquid on the high level sensor generates a signal, which causes the solenoid-operated drain valve 102 to again open and to start the pump 100. This cycle continues until the operator presses the “Stop Suction” operation 212 on the touch screen 20 or until an alarm condition interrupts the vacuum.

It should be appreciated that the pump 100 is responsive to the high level sensor to prevent overfilling of the reservoir which could cause the fluid waste to enter the facility's vacuum lines. The pump is also responsive to the low level sensor so that it shuts off prior to the reservoirs being completely drained so as to prevent air from entering the drain line 98. If air entered the drain line, the drain line would not be full thereby adversely affecting the accuracy of the Fluid Pumped display 200 which is calculated based on pump cycles assuming the drain lines are full of fluid at all times.

It should also be appreciated that it may be possible for the incoming fluid rate to reach an equilibrium with the pump discharge rate, whereby the drain valve 102 will remain open and the pump 100 will continue to operate until the incoming fluid rate is eventually reduced to where the fluid is again drawn down to the low level sensor 80, at which point the drain valve 102 closes and the pump 100 is stopped until the fluid level again reaches the high level sensor 82.

In the unlikely event that the high level sensor 82 is faulty and fails to actuate the pump 100 such that the fluid level reaches the maximum level sensor 84, the maximum level sensor preferably generates a signal to actuate the vacuum interrupt valve 94 to close off the vacuum line 90 until the problem is resolved. As a second layer of safety to prevent liquid from entering the facility's vacuum system, in the very remote event that both the high-level sensor 82 and the maximum level sensor 84 are inoperative, such that fluid is forced into the vacuum line 90, as soon as the fluid comes into contact with the filter 92, the filter 92 immediately swells preventing the passage of liquid and air which in turn causes solenoid-operated vacuum interrupt valve 94 to actuate to interrupt the vacuum.

As the pump 100 operates, the volume of fluid passing through the pump is displayed in the Fluid Pumped display 200 of the touch screen 20 and is preferably stored in memory for later retrieval and display or for outputting electronically to another device such as USB flash drive or other portable data storage device, a printer, etc.

As previously identified, upon completion of the surgical procedure and removal of the suction tubing 88 from the surgical site so fluid is no longer being aspirated, the operator selects the “Stop Suction” operation 212 using the touch screen 20 thereby causing the vacuum interrupt valve 94 to actuate and close-off the vacuum source from the vacuum lines 90. Additionally, the low-level sensor 80 is bypassed so that the pump 100 continues to pump all remaining fluid from the reservoirs 50, 52. The system 10 is preferably calibrated so that the amount of fluid remaining in the reservoirs 50, 52 below the low level sensor 80 and the drain line 98 upstream of the pump 100 is added to the Fluid Pumped value so that this additional volume is properly taken into account on the Fluid Pumped display 200.

Shortly upon pressing the Stop Suction operation, the operator is preferably prompted on the touch screen display 20 to select the “Start Clean Cycle” operation 214. Upon pressing the Start Clean Cycle operation, the operator is preferably instructed on the screen display 20 to remove the suction hose 88 from the suction port 26 and to place the cleaning solution bottle 30 onto the hanger 28 and to attach the cleaning solution tube 120 to the to the suction port 26. In a preferred embodiment, the cleaning solution tube 120 is attached to a reusable cap that can be attached to the cleaning solution bottles 30 as they are used. The hanger 28 is preferably formed to receive the contours or shape of the bottle or other packaging 30 so the package is more stably held or supported.

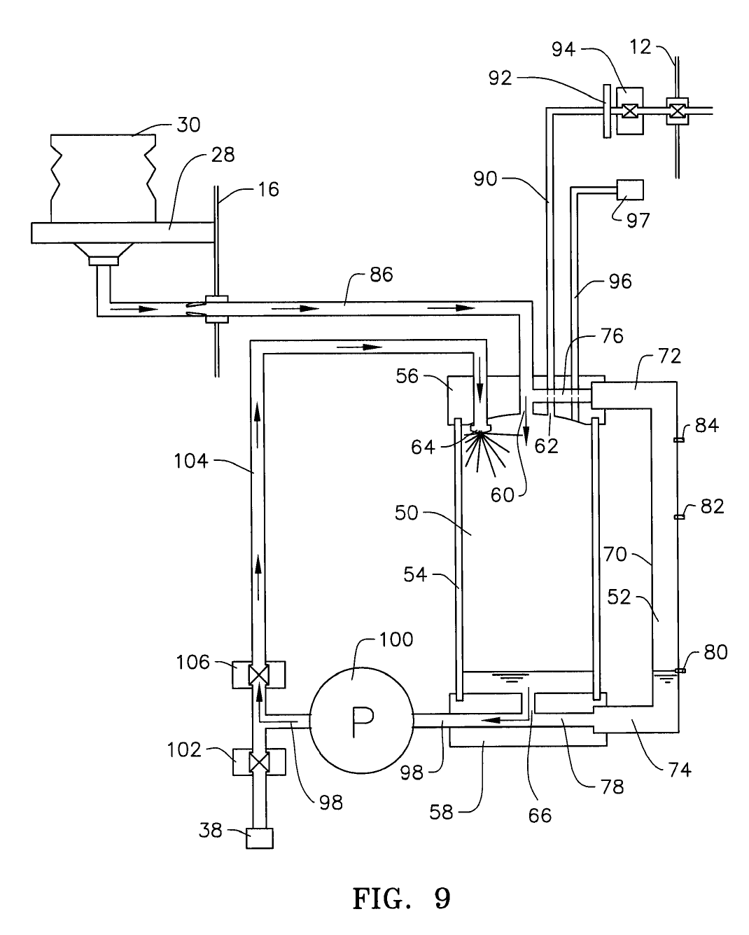

Upon pressing the Start Clean Cycle, the vacuum interrupt valve is again actuated to open the vacuum line 90 again producing a negative pressure in the first reservoir 50. As illustrated in FIG. 9, due to the negative pressure in the reservoir 50 the cleaning solution is drawn out of the bottle 30 and into the reservoir. The bottle is preferably designed to collapse as the cleaning fluid is drawn out of the bottle by the negative pressure to ensure that the bottle is for a single use only. The volume of the bottle 30 is preferably matched to fill the reservoirs 50, 52 just up to the maximum level sensor 84. Preferably, during the Clean cycle, the low, high and maximum level sensors 80, 82, 84 are disabled or bypassed. Also, preferably, the drain valve 102 is opened and the pump 100 momentarily started so that any aspirated fluid in the drain line 98 is flushed.

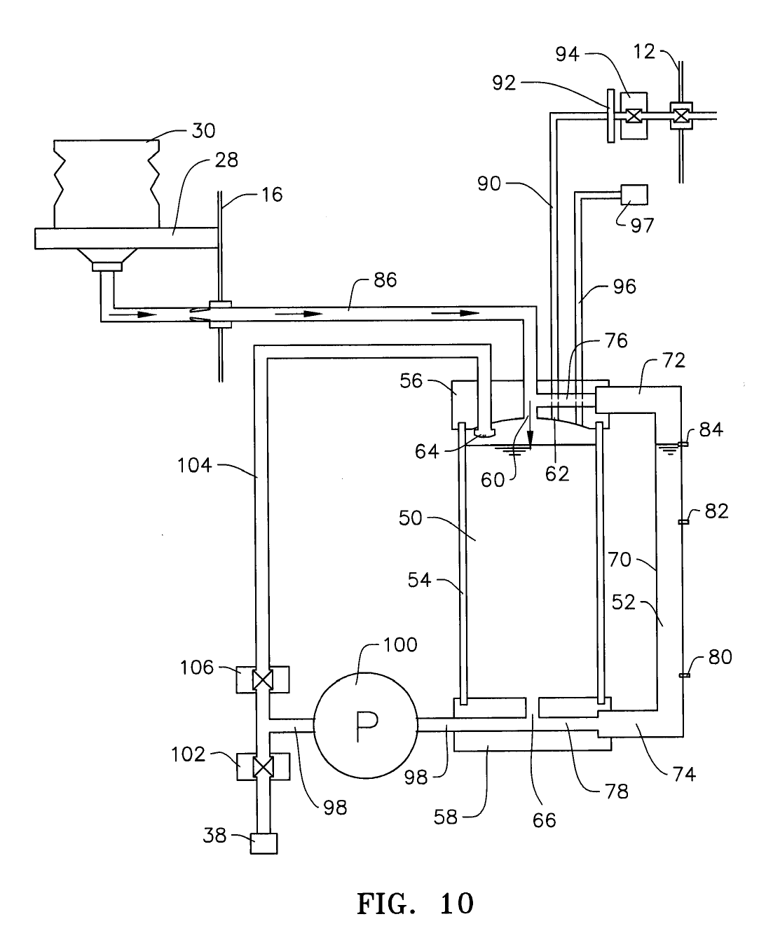

During the Clean cycle, the cleaning solution is preferably held in the reservoirs for a predetermined time period. After this predetermined dwell time or presoak, the normally closed solenoid-operated recirculation valve 106 is opened and the pump 100 is actuated. The drain valve 102 remains closed. Thus, as illustrated in FIG. 10, the cleaning solution is caused to be pumped through the recirculation line 104 and back into the reservoir 50 through the recirculation nozzle 64. The recirculation nozzle 64 is preferably configured to spray the cleaning solution against the cylinder walls 54 of the first reservoir 50. This recirculation process continues for a predetermined period of time until the cylinder walls are thoroughly cleaned and disinfected. The pre-soak period and recirculation period may vary depending on the cleaning solution used. The cleaning solution may be any suitable disinfectant. However, the preferred cleaning solution is a shelf-stable, pH-neutral, oxychlorine compound, such as Microcyn® which not only disinfects but also assists in breaking down biological materials in the aspirated fluid. Microcyn is distributed by Oculus Innovative Sciences, 1129 North McDowell Blvd., Petaluma, Calif. 94954.

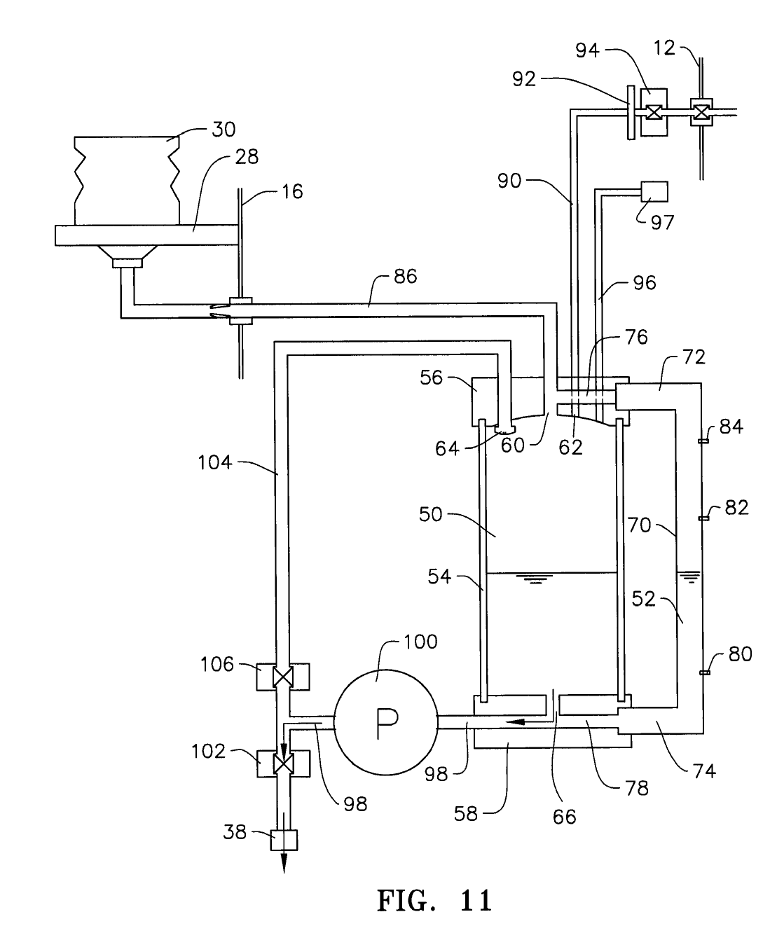

As illustrated in FIG. 11, after the predetermined recirculation period, the recirculation valve 106 is closed, the drain valve 102 is opened and the pump 100 is actuated to pump the cleaning solution from the reservoirs 50, 52. The cleaned and disinfected system 10 is now again ready for use. Pumping the cleaning solution during Clean cycle does not increase the Fluid Pumped display 200.

It should be appreciated that instead of a bottle 30, the cleaning solution may be provided in a collapsible, single use bag, similar to an intravenous (IV) bag, and the hanger 28 may be configured in any suitable manner to support the bag. Alternatively, rather than utilizing disposable, single use, bottles or bags, a cleaning solution reservoir may be provide inside or external to the housing 12. In such a system, the PLC may be programmed to automatically dispense the predetermined volume of cleaning solution from the cleaning solution reservoir into the first reservoir 50.

In yet another alternative embodiment, the cleaning solution (or a separate sterilizing solution) may be disposed to be in fluid communication with the first reservoir during the normal operation of the system instead of only during the cleaning cycle. The cleaning/sterilizing solution may be provided in disposable bottles or bags or an internal or external disinfecting solution reservoir as previously described. The controller may be programmed to periodically and/or continuously dispense the cleaning/sterilizing solution into the first reservoir 50, via gravity, negative pressure or via a pump, at the same time as the aspirated fluid enters the first reservoir 50 to immediately destroy any pathogens and or accelerate the breakdown of the biological material in the aspirated fluid before being pumped into the sanitary sewer.

The system 10 may include a radio frequency identification (RFID) transceiver (not shown) which communicates with an RFID tagged cleaning solution bottle or bag 30 to ensure compliance with proper cleaning practices and warranty provisions. For example, the PLC of the system 10 may be programmed to prevent the “Suction Start” operation from being performed unless the system had previously performed a Clean Cycle using a recognized RFID tagged product. The PLC may also be programmed to recognize a unique RFID tag only once, so the same bottle or bag 30 cannot be refilled with a non-approved cleaner and then reused. Additionally the PLC may be programmed to accept only certain RFID tagged products produced within a certain date range to ensure that the cleaning solution has not exceeded its shelf-life.

The foregoing description is presented to enable one of ordinary skill in the art to make and use the invention and is provided in the context of a patent application and its requirements. Various modifications to the preferred embodiment of the apparatus, and the general principles and features of the system and methods described herein will be readily apparent to those of skill in the art. Thus, the present invention is not to be limited to the embodiments of the apparatus, system and methods described above and illustrated in the drawing figures, but is to be accorded the widest scope consistent with the spirit and scope of the appended claims.

1. A system for continuously aspirating a fluid from a patient, comprising:
a first reservoir in communication with a vacuum source producing a negative pressure therein, said first reservoir having a fluid inlet through which the fluid from the patient is drawn therein by said negative pressure therein, said first reservoir further having a fluid outlet;
a second reservoir in communication with said first reservoir;
a pump in communication with said fluid outlet operable to draw fluid from said first and second reservoirs while additional fluid from the patient continues to be drawn into said first reservoir through said fluid inlet.

2. The system of claim 1 wherein said pump is operatively responsive to sensors disposed to detect predefined high fluid level limits and low fluid level limits in said second reservoir.

3. The system of claim 1 wherein said pump is a metering pump.

4. The system of claim 1 wherein said metering pump is a positive displacement pump.

5. The system of claim 1 wherein said positive displacement pump is a peristaltic pump.

6. The system of claim 1 further comprising a cleaning solution volume disposed for communication with said first reservoir.

7. The system of claim 6 wherein said cleaning solution volume is substantially equal to but less than a said first and second reservoir volumes.

8. The system of claim 6 wherein said cleaning solution volume is provided in a single-use disposable package.

9. The system of claim 6 wherein said cleaning solution is a shelf-stable, pH-neutral, oxychlorine compound.

10. The system of claim 8 wherein said cleaning solution is a shelf-stable, pH-neutral, oxychlorine compound.

11. The system of claim 8 wherein said system further comprises a hanger disposed to removably support said single-use disposable package.

12. The system of claim 1 wherein said first and second reservoirs and said pump are disposed within a secured housing; said housing have a window through which said aspirated fluid received within said first reservoir is viewable.

13. The system of claim 12 further including lighting disposed to back-light said first reservoir within said housing such that the aspirated fluid is better viewed through said window.

14. A method for continuously aspirating and collected fluid waste during a medical procedure without interruption for disposal of the collected fluid waste, said method comprising the steps of:
providing a first reservoir in communication with a vacuum source producing a negative pressure therein, said first reservoir having a fluid inlet and a fluid outlet;
providing a second reservoir in communication with said first reservoir;
providing a suction hose operably in communication with said fluid inlet at one end and with the fluid waste at a distal end, said negative pressure drawing the fluid waste through said suction hose and into said first reservoir;
pumping the fluid waste from said first and second reservoirs while additional fluid waste continues to be drawn into said first reservoir through said fluid inlet.

15. The method of claim 14 wherein said step of pumping is initiated upon said fluid waste in said second reservoir reaching a predefined high fluid level.

16. The method of claim 15 wherein said step of pumping is suspended upon said fluid waste in said second reservoir reaching a predefined low fluid level and until said fluid waste in said second reservoir again reaches a predefined high fluid level.

17. The method of claim 14 further comprising the step of measuring fluid waste volume pumped using a metering pump.

18. The method of claim 14 further comprising the step of measuring fluid waste volume pumped using a positive displacement pump.

19. The method of claim 14 further comprising the step of measuring fluid waste volume pumped using a peristaltic pump.

20. The method of claim 14 further comprising the step of cleaning after the medical procedure by drawing under said negative pressure a volume of cleaning solution through said fluid inlet.

21. The method of claim 20 wherein said step of cleaning further comprises recirculating said volume of cleaning solution by pumping said volume of cleaning solution from said first and second reservoirs and back into said first reservoir through said fluid inlet.

22. The method of claim 21 wherein said step of cleaning further comprises pumping said volume of cleaning solution from said first and second reservoirs.

23. The method of claim 20 wherein said volume of cleaning solution is drawn from a single-use disposable package.
